(19) United States
(12) Patent Application Publication
(10) Pub. No.: US 2023/0337761 A1
(43) Pub. Date: Oct. 26, 2023

(54) PROTECTIVE DEVICE FOR ONE HAND

(71) Applicant: Jakob ROGALSKI, Bedburg (DE)

(72) Inventor: Jakob ROGALSKI, Bedburg (DE)

(21) Appl. No.: 18/137,104

(22) Filed: Apr. 20, 2023

(30) Foreign Application Priority Data
Apr. 26, 2022 (EP) 22170087.5

(51) Int. Cl. A41D 13/08

(52) U.S. Cl. CPC A41D 13/082; A41D 13/087

(57) ABSTRACT

The invention relates to a protective device for a hand, which comprises precisely two finger protection elements and a palm protection element connecting the two finger protection elements, wherein one finger protection element is intended for the thumb and the other finger protection element is intended for a further finger, in particular the index finger, of a user, and the palm protection element is designed to be planar, so that it only comes into contact with the palm of a user's hand, whereas the back of the user's hand remains free or essentially free.

### CROSS-REFERENCE TO RELATED APPLICATIONS AND CLAIM TO PRIORITY

This application relates to application No. 22170087.5 filed Apr. 26, 2022 in the European Patent Office, the disclosure of which is incorporated herein by reference and to which priority is claimed.

### FIELD OF THE INVENTION

The invention relates to a protective device for a hand, which comprises exactly two finger protection elements and a palm protection element connecting the two finger protection elements.

### BACKGROUND OF THE INVENTION

A high proportion of reported accidents in health services and laboratories are due to needlestick injuries, cut injuries and scratch injuries to the skin caused by piercing or cutting instruments. The Berufsgenossenschaft für Gesundheitsdienst and Wohlfahrtspflege (BGW) cites as one cause that pointed and sharp instruments such as cannulas, lancets, needles, scalpels and the like are not disposed of correctly immediately after use. In some cases, users fail to reseal with associated securing elements, such as caps or sleeves, and dispose of the instruments openly in a waste container, which then poses a significant health risk.

“Fingerlings” are known which can be placed individually on the fingertips. However, these finger cots are often made of inadequate material, so that they do not provide adequate protection against puncture and cut injuries in the finger area. Protection limited to the fingers is also considered insufficient in some cases.

DE 43 17 108 A1 shows a surgical finger guard as a needle puncture protection. This finger guard has an element which is flexible and resistant to a needle point. The element is configured to conform to a finger to provide a continuous barrier to a needle stick over at least a portion of the finger. The element has a continuous, undulating shape that terminates with an end cap which is resistant to a needle point. The disclosed elements extend over individual fingers, analogous to fingerlings.

U.S. Pat. No. 4,864,661 A shows a surgical glove made of a thin elastic material which is designed to be resistant to nail punctures in selected areas on the inside of the hand. For this purpose, it comprises suitable materials in the areas, for example a woven material or a solidly designed material.

EP 0 320 541 A1 discloses another surgical glove that is resistant to needlesticks. It comprises a thin base glove made of latex or a synthetic rubber, provided on the inside with a reinforcing material with a tightly woven puncture-resistant reinforcing material to minimize the risk of punctures or tears.

DE 20 2006 010 706 U1 discloses fingerlings and a partial glove for preventing cuts in hairdressing. The partial glove covers two of the user's fingers, specifically the index and middle fingers, the palm, the back of the hand and the wrist. It is made of a cut-resistant nitrile plastic or a metallic fabric covered with plastic.

The protective devices known from the prior art have proven themselves in principle to protect the hands of users from injuries caused by pointed and sharp objects. However, there is still a need for improved devices. In particular, there is a need for protective devices that allow very safe and at the same time rapid and convenient disposal of pointed or sharp medical objects after use.

### SUMMARY OF THE INVENTION

It is therefore the task of the present invention to specify a protective device of the type mentioned at the beginning, which protects the user particularly reliably against injuries in the area of the hand when handling pointed or sharp objects, in particular when disposing of them, and which at the same time is particularly easy and convenient to use.

This task is solved in a protective device of the type mentioned above in that one finger protection element is intended for the thumb and the other finger protection element is intended for a further finger, in particular the index finger, of a user, and the palm protection element is designed to be flat so that it only comes into contact with the palm of a user's hand, whereas the back of the user's hand remains free or essentially free.

In other words, when worn on the hand as intended, the protective device according to the invention—in contrast to conventional “full gloves”—covers only part of the hand, specifically two fingers at least in sections and the palm. The fingers protected by the device according to the invention are the thumb and another finger, preferably the index finger. The structure is oriented to the typical hand movements that are carried out above all when closing or re-closing pointed and sharp medical utensils, such as cannulas, lancets, scalpels and the like. The securing or closing elements, which may be sheaths or caps, for example, are generally held between the thumb and another finger, particularly the index finger, while the previously used instrument is carefully inserted. The applicant has found that in this process, especially the fingertips of the two fingers used for holding, but also the palm of the user's hand, are at risk of being injured, for example as a result of slipping or incorrect “aiming” during the insertion process. The device according to the invention reliably protects precisely these particularly vulnerable areas of the hand during the reclosure process. At the same time, the protective device according to the invention can be applied very conveniently and particularly quickly by a user. He only has to insert the tips of his thumb and another finger, in particular his index finger, into the respective finger protection element. The palm protection element connecting the two finger protection elements then lies protectively in front of his palm.

Since the protective device according to the invention can be put on particularly quickly, especially in comparison with previously known full as well as partial gloves, which have to be pulled over the hand and up to the wrist in a relatively laborious manner, the effort associated with disposal is minimized. If the effort of proper as well as safe disposal is low, users are more likely to opt for it and the risk of health hazards is reduced. The protective device according to the invention can also be used in combination with thin disposable rubber gloves, which are often worn in the medical or nursing field. These do not have to be removed before the device according to the invention is put on. This will be the case with the previously known full or partial gloves with a protective function against punctures and cuts, since it is problematic to put on another full or partial glove over an existing rubber glove. Since the protective device according to the invention does not require the disposable gloves to be removed, the effort involved can be further reduced.

The fact that one of the finger protection elements is intended for the thumb and the other for a further finger, in particular the index finger, of the hand of a user means in particular or includes in particular that the elements are designed and arranged in such a way that one can be placed on the thumb and the other on the further finger, in particular the index finger, of the hand of a user. If the two finger protection elements are placed on the fingers, the protective device according to the invention is worn in the intended manner.

In an advantageous embodiment of the protective device according to the invention, it is provided that the finger protection elements both lie closer to one end region of the palm protection element than to another end region of the palm protection element opposite the one end region. In particular, the two finger protection elements may be located directly at the one end region of the palm protection element. Alternatively or additionally, it can be provided that the other end region, i.e. the end region at which the finger protection elements do not lie or from which they are further away, is a free end region of the palm protection element.

The palm protection element is preferably tongue-shaped.

It may be provided that the palm protection element has an end region with a U-shaped outer contour, called the tip region, and an end region opposite the tip region, called the root region. Then, the finger protection elements are both preferably closer to the root region than to the tip region. They may be located immediately adjacent to the root region.

The tip region is preferably a free end region of the palm guard element.

It is particularly preferred that the root area ends with a particularly straight edge opposite the tip area and that the finger protection elements extend to this edge. In other words, the palm protection element then ends in the area of the finger protection elements or extends starting from the finger protection elements. This design has proven to be particularly suitable, especially in the event that one of the finger protection elements is intended for the index finger. There is then an ideal adaptation to the anatomical structure of the human hand, in which the thumb and index finger are both located near or at one end region of the palm. On the left hand, looking at the palm, they are specifically located at the left end region of the palm, and on the right hand they are located at the right end region.

Furthermore, it may be provided that the two finger protection elements are arranged laterally of the palm protection element with respect to the two opposite end regions of the palm protection element, preferably projecting laterally from the palm protection element. With respect to the two opposite end regions laterally means in particular with respect to an imaginary line connecting these two end regions laterally of the palm protection element.

It has also proved particularly advantageous if one of the two finger protection elements is arranged on each of two opposite sides of the palm protection element.

Alternatively or additionally, it can be provided that the finger protection elements are open at one end and closed at their other end opposite the open end, the open end facing the palm protection element and the closed end facing away from the palm protection element. It is then possible to insert the tips of the thumb and another finger, in particular the thumb and index finger, of a hand into the finger protection elements in a particularly convenient manner in order to put on the protective device according to the invention. For this purpose, the thumb and index finger can be positioned apart in a V-shape, for example.

The palm protection element then lies between the two finger protection elements and its one end region, in the worn state, lies in particular on that end region of the palm of a user's hand which connects the thumb and index finger to one another and, starting from there, preferably extends over the entire palm of the user's hand.

In another particularly preferred embodiment of the protective device according to the invention, it is provided that the finger protection elements each comprise a finger protection cap with a closed upper side and an open lower side. It is also possible that the finger protection elements are formed by such finger protection caps. Finger protection caps can be placed on the thumb and the further finger of a user in a simple manner and, in particular, reliably protect the tips of these fingers.

The finger protection caps are preferably characterized at least substantially by the shape of a hollow truncated cone or at least substantially by the shape of a hollow cylinder closed on one side. These shapes have proved particularly suitable for caps which can be placed on fingers. By at least substantially it is to be understood that there may be deviations from the exact hollow truncated cone or hollow cylinder shape. For example, the finger protection caps can in particular be rounded, e.g. have rounded edges and curved, in particular convex end walls, as is known from thimbles.

It is also possible for the finger protection caps to be characterized by a greater material thickness in their end faces than in the remaining area. The reinforcement of the material on the end faces provides particularly high resistance in risk zones. When worn, the fingertips are located under the end faces and are particularly at risk, especially with regard to punctures.

A further embodiment of the invention is characterized by the fact that the finger protection elements have a finger protection web. If such a web is present, it is connected in particular to the open underside of the finger protection cap of the respective finger protection element. In particular, a finger protection web can be used to protect a lower section of the thumb and other finger, in particular the index finger, which is closer to the palm of the hand.

In this embodiment, the upper sections of the thumb and the other finger are covered and protected in particular all around, while the lower sections of the fingers are protected only on their inside by the respective finger protection web, but remain free on the outside or top and laterally. The finger protection webs are accordingly preferably designed and arranged to cover and thus protect a longitudinal section of the respective finger, in particular only on the inside. The finger protection webs are preferably flat and come into contact only on the inside of the fingers, while leaving the outside, top and sides of the fingers free.

If the finger protection elements each comprise a finger protection web, it is preferred that the finger protection web of the finger protection element intended for the thumb is shorter than the finger protection web of the finger protection element intended for the other finger, in particular the index finger.

A further particularly preferred embodiment is further characterized by the fact that the finger protection elements are connected to one another exclusively via the palm protection element and, in particular, are formed in one piece. If the finger protection elements comprise finger protection caps and adjoining finger protection webs, the palm protection element is preferably connected to the finger protection webs.

In addition, a fastening strap can be provided, one end of which is firmly connected to the palm protection element, preferably in the region which, in the intended state, is located between the root of the thumb and the root of the further finger, and the free end of which can be releasably fixed to the palm protection element, in particular to the region thereof which is associated with the palm of the hand of a user, for example by means of a Velcro fastener, after the fastening strap has been placed over the back of the hand of the user. In this way, the palm protection element can be additionally fixed to the hand.

Nubs can be provided on the inside and/or outside of the finger protection caps, enabling particularly safe handling of pointed or sharp utensils. Nubs provided on the inside of the finger protection caps ensure above all a particularly secure hold of the device according to the invention or of the finger protection caps on the hand of a user. The nubs on the outside primarily allow more control over the objects to be handled.

The material thickness of the protective device according to the invention is suitably selected to ensure a sufficient degree of stab or cut resistance.

It has proved particularly suitable if the material thickness of the finger protection caps is at least 1.0 mm, in particular at least 2.0 mm, at least in sections and/or the material thickness of the finger protection caps is at most 5.0 mm, in particular at most 4.0 mm, and is preferably 3.0 mm.

Alternatively and/or additionally, it may be provided that the material thickness of the finger protection webs, at least in the region of the front ends associated with the fingertips, is at least 1.0 mm, in particular at least 2.0 mm, and/or is at most 5.0 mm, in particular at most 4.0 mm, and is preferably 3.0±0.5 mm.

In a further embodiment of the invention, the interior of the finger protection caps can have a diameter of 1.5 to 3.0 cm in particular at their widest point and/or a length in the range of 1.5 to 6.0 cm. It is particularly preferred if the finger protection caps are designed in such a way that they only cover the respective front thumb or finger limb up to the first finger joint. In this case, the finger protection caps have a length of at least 2.0 cm, in particular at least 2.5 cm, and/or at most 4.0 cm, in particular at most 3.0 cm.

The palm protection element is expediently designed and arranged in such a way that it extends protectively in front of the palm of the user's hand when the protective device is worn as intended and the two finger protection elements are each placed on a finger, in particular the thumb and index finger of a user.

It can be designed as a flexible mat that lies between the two finger protection elements and, when worn, covers the palm of the hand and rests against it.

The protective device according to the invention and/or its finger protection elements and/or palm protection element preferably comprise or consist of an elastically deformable material, in particular at least one elastomer or silicone. Alternatively or additionally, the finger protection elements and/or the palm protection element may comprise or consist of latex. It is important that the palm protective element be sufficiently flexible so that it conforms to the palm of the hand during use. Similarly, the finger protectors must be flexible enough to allow free movement of the thumb and other finger. This is ensured by matching the elasticity of the material used and the thickness of the material. The material may be reinforced. In particular, reinforcing elements such as strips of metal sheet, plastic or the like, or reinforcing fibers can be embedded in the material.

A further embodiment of the invention is characterized in that the palm protection element has a thickness of at least 1.0 mm, in particular at least 2.0 mm and/or a maximum of 5.0 mm, in particular a maximum of 4.0 mm, the thickness preferably being 3.0±0.5 mm, and/or in that the palm protection element has a length of at least 6.0 cm, in particular at least 7.0 cm and/or of at most 11.0 cm, in particular at most 9.0±0.5 cm.

In a further practical embodiment, the protective device according to the invention is designed in such a way that it leaves three fingers, preferably the ring finger, middle finger and little finger, and/or the back of the hand and/or the wrist free when a user wears it in the intended manner. It has been found to be quite particularly suitable if it leaves three fingers, in particular the ring finger, middle finger and little finger, and the back of the hand and wrist free. Particularly preferably, only the upper end regions of two fingers are covered by the two finger guards and the palm is covered by the palm protection element.

With regard to the embodiment of the invention, reference is also made to the subclaims and to the following description of an embodiment with reference to the accompanying drawing.

### BRIEF DESCRIPTION OF THE DRAWINGS

In the drawings, shown in a purely schematic representation:

FIG. 1 an example of a protective device according to the invention when worn on the user's hand as intended, in a view to the palm of the hand;

FIG. 2 the protective device from FIG. 1 in the worn state in a view to the back of the hand:

FIG. 3 the protective device of FIG. 1 in the worn state in a view of the palm of the hand, with the thumb and index finger and the finger protective caps placed on them brought together to hold an object;

FIG. 4 the protective device of FIG. 1 in the worn state in a view to the palm of the hand, with the thumb and index finger and the finger protective caps placed on them brought together to hold an object;

FIG. 5 the protective device from FIG. 1 in the worn state from the side;

FIG. 6 the protective device of FIG. 1 in the worn state in a view to the palm of the hand, with the tips of the user's middle finger, ring finger and little finger positioned behind the palm protective element;

FIG. 7 the view from FIG. 6, with the thumb and index finger brought closer together;

FIG. 8 the protective device of FIG. 1 in the unworn, unfolded state in plan view; and

FIG. 9 a sectional view of the protective device along the line AA in FIG. 8.

In the figures, the same components or elements are marked with the same reference signs.

### DETAILED DESCRIPTION OF THE PREFERRED EMBODIMENT(S)

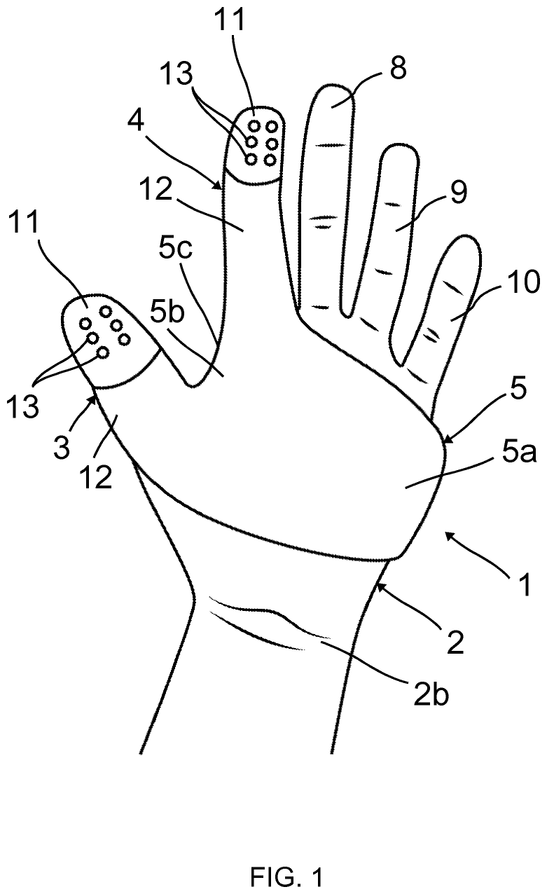

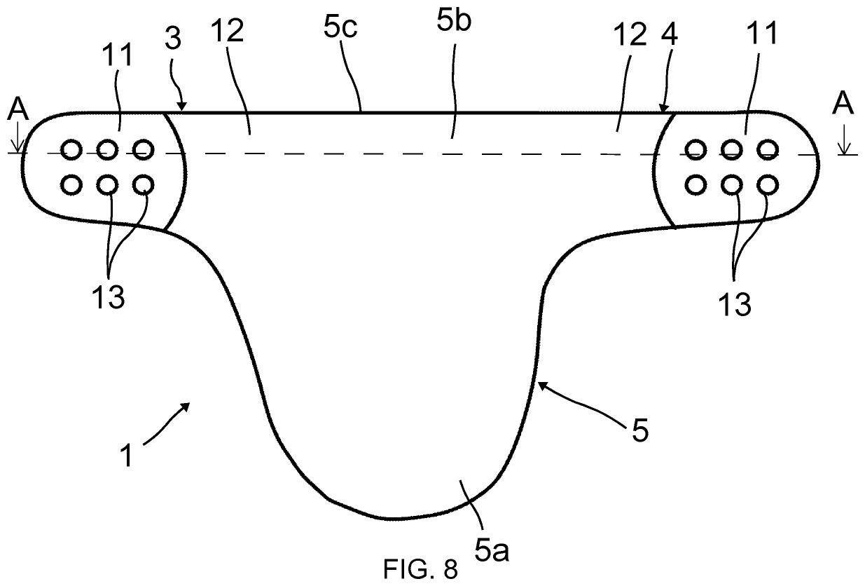

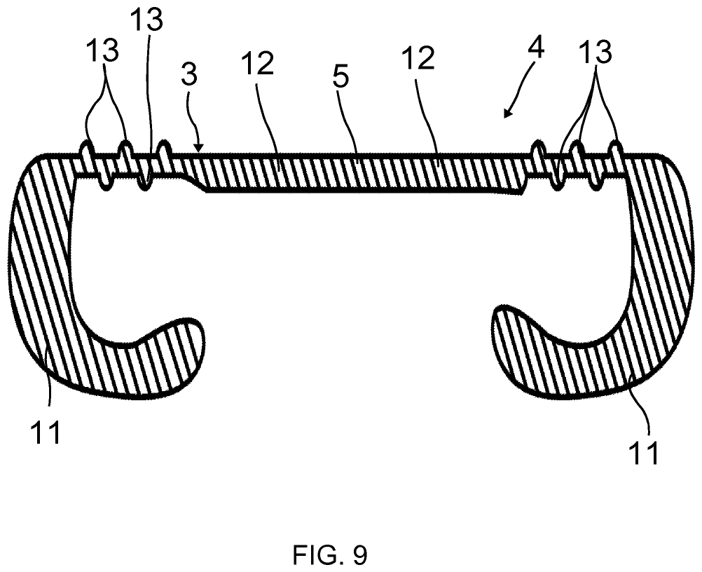

FIGS. 1 to 9 show various views of an embodiment of a protective device 1 according to the invention, with FIGS. 1 to 7 showing the device 1 in the state worn on the hand 2 of a user, while it can be taken from FIGS. 8 and 9 in the unworn state in the solo position.

The protective device 1 according to the invention comprises two finger protection elements 3, 4 and a palm protection element 5 which connects the two finger protection elements 3, 4 to one another. Thereby, one finger protection element 3 is recognizably intended for the thumb 6 and the other finger protection element 4 is recognizably intended for the index finger 7 of the hand 2 of a user.

The protective device 1 comprises exactly two finger protection elements 3, 4. For the remaining fingers, i.e. the middle finger 8, ring finger 9 and little finger 10, the protective device 1 has no finger protection elements. Accordingly, these fingers 8, 9, 10 remain free when the device 1 is worn on the hand 2 of a user. The same applies to the back of the hand 2a and the wrist 2b, which also remain free. The protective device 1 has no protective elements for these either.

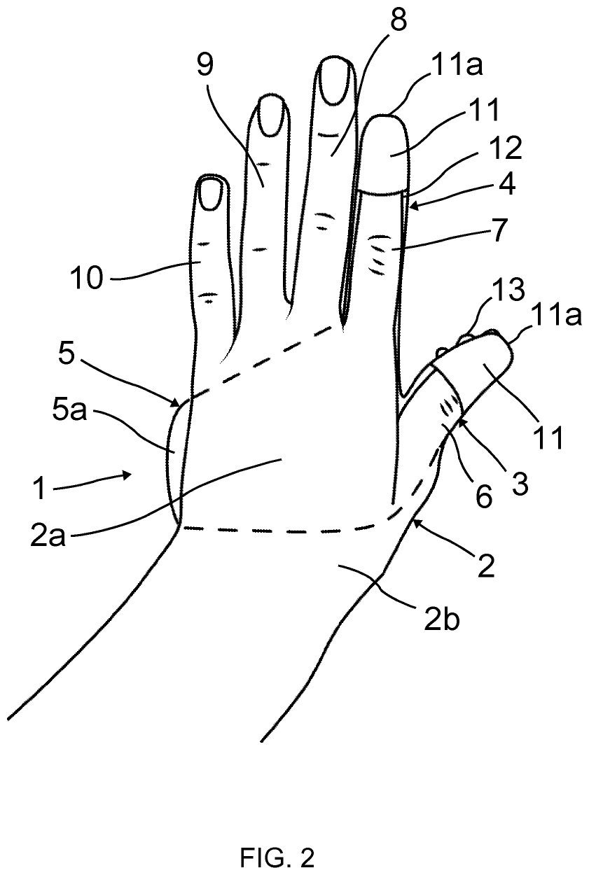

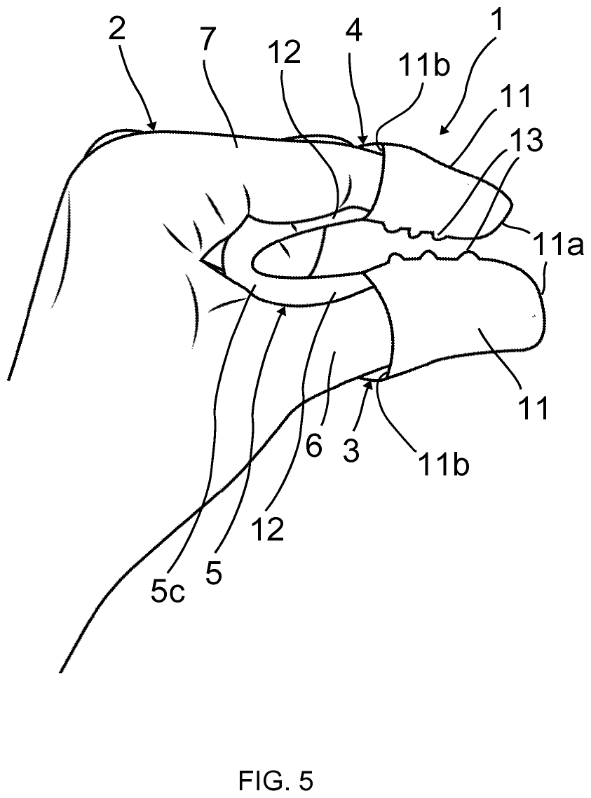

In the embodiment shown, the two finger protection elements 3, 4 for the thumb 6 and the index finger 7 each comprise a finger protection cap 11 and a finger protection web 12. The finger protection caps 11 correspond in shape to conventional thimbles which are also used in sewing. They have approximately the shape of hollow truncated cones, the front end section of which is recognizably rounded and thus adapted to the shape of the fingertips to be accommodated. The caps 11 are closed at their front side 11a and open at their underside 11b opposite the front side, so that they can be placed on the fingertip (cf. in particular FIGS. 2 and 5, which show the placed-on state particularly well).

As can be seen, the length of the finger protection caps 11 is selected in such a way that, when worn, only an upper section of the thumb 6 or index finger 7 is received and covered by them, specifically only that section which extends from the tip of the finger to approximately the uppermost joint of the respective finger 6, 7. The remaining portion from the thumb and index finger to the palm is left free by the finger protection caps 11. The length of the interior of the finger protection caps 11 is 3.0 cm here. The diameter of their interior at the widest point is 2.5 cm. These values are to be understood as purely exemplary. Conveniently, the protective device 1 according to the invention—just like conventional gloves—is used in different sizes for hands of different sizes.

Connected to the finger protection caps 11 in a downward direction, in other words beginning at their open lower sides, is in each case a finger protection web 12 which protects the thumb 6 and index finger 7 over the remaining longitudinal section of the finger 6, 7 which is not covered by the finger protection cap 11 and extends to the metacarpus, but only in the region of the inner surface of the respective finger 6, 7. They are appropriately configured and arranged to cover and protect the lower longitudinal portion of the respective finger 6, 7 extending from the uppermost finger joint to the lower end of the finger 6, 7 on the inside only. On the outside or rear side and laterally, they leave the thumb 6 and index finger 7 free. The finger protection webs 12 here have an approximately rectangular outer circumference. They both have a width of 2.0 cm, but have different lengths. The finger protection web 12 of the finger protection element 3 intended for the thumb 6 is shorter than the finger protection web 12 of the finger protection element 3 intended for the index finger 7, in order to take account of the different lengths of these two fingers.

The finger protection webs 12 are formed in one piece with the finger protection caps 11 and are molded onto them. The finger protection webs 12 and finger protection caps 11 are made of an elastic plastic material, in particular an elastomer or silicone. They have a suitable material thickness to provide good protection against punctures, scratches, cuts and the like. In the example shown, the material thickness in the region of the end faces 11a of the finger protection caps 11 is 5.0 mm, while in the remaining regions of the finger protection caps 11 it is 3.0 mm. The end faces 11a of the finger protection caps 11 are thus reinforced in order to provide particularly good protection in the risk zones there. The greater material thickness can be seen in the sectional view in FIG. 9.

The material thickness of the finger protection bars 12 is 3.0 mm.

The finger protection elements 3, 4 have nubs 13 in the areas of the finger protection caps 11 adjacent to the finger protection webs 12. The nubs 13 are provided both on the inside and on the outside of these areas of the finger protection caps 11. On the outside, the nubs 13 are positioned on that side of the finger protection caps 11 which is also adjoined in each case by the finger protective webs 12 and which faces the palm protection element 5. On the inner side, nubs 13 are positioned opposite the outer nubs 13, but laterally offset from them. In the areas where nubs 13 are provided, the material thickness of the finger protection elements 4 is somewhat reduced, so that the tips of the inner nubs 13 are approximately cohesive with the inner side of the finger protection webs 12. The inner nubs 13 ensure above all a particularly secure hold of the device according to the invention or of the finger protection caps 11 on the hand 2 of a user, and the outer nubs 13 above all more control as far as objects to be handled are concerned.

The palm protection element 5, which here is tongue-shaped, connects the two finger protection elements 3, 4 of the device 1, specifically their finger protection webs 12, to each other. The finger protection elements 3, 4, i.e. the finger protection webs 12 of these are connected to each other exclusively via the palm protection element 5.

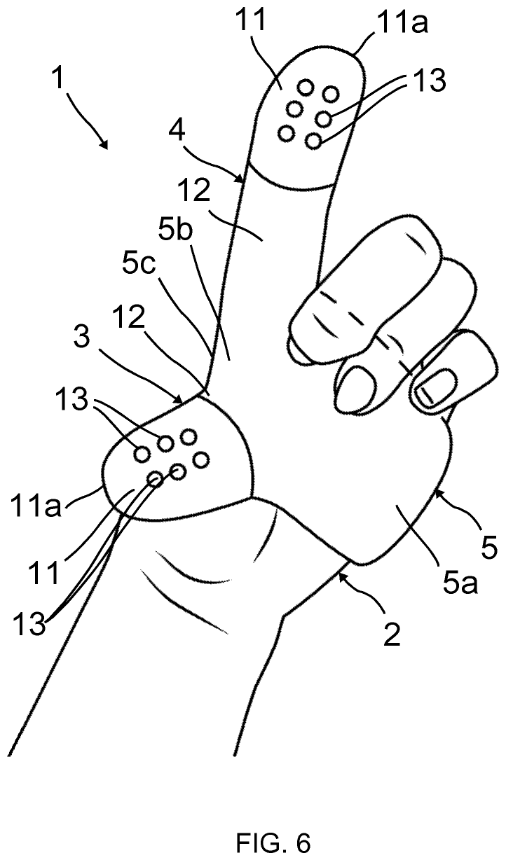

The palm protection element 5 is designed as a flexible mat. It has a free end region with a U-shaped outer contour, a tip region 5a, and an end region opposite the tip region 5a, root region 5b (see in particular FIGS. 6 and 8). The two finger protection elements 3, 4 are both located closer to the root region 5b than to the tip region 5a. In the example shown, they lie immediately adjacent to the root region 5b. The root region 5b terminates with a straight edge 5c opposite the tip region 5a, and the finger protection elements 3, 4, specifically their finger protection webs 12, extend to this edge 5c.

As can be seen in the figures, the two finger protection elements 3, 4 further lie to the side of the palm protection element 5 with respect to the two opposite end regions 5a and 5b, one on each of two opposite sides thereof. Here, they project laterally from the palm guard element 5. The two finger protection elements 3, 4 are in alignment.

The open end of the finger protection elements 3, 4 and the open end of the respective finger protection caps 11 face the palm protection element 5, and the closed end faces of the finger protection elements 3, 4, specifically of their finger protection caps 11, face away from the palm protection element 5. In this way, the tips of the thumb 6 and index finger 7 can be inserted into the finger protection elements 3, 4 and their caps 11 in a particularly convenient manner in order to put on the protective device 1.

As can be seen in the figures, the palm protection element 5 is designed and arranged in such a way that, when the protective device 1 is worn as intended and the two finger protection elements 3, 4 or their caps 11 are placed on the thumb 6 and index finger 7 of a user, it extends protectively in front of the palm of the user's hand. Here, it has a length of 8.0 cm (extending from the edge 5c to the end of the tip area 5a).

The palm protection element 5 and the finger protection elements 3, 4 are formed in one piece. The palm protection element 5 is made of the same material as the finger protection elements 3, 4 and is 3.0 mm thick.

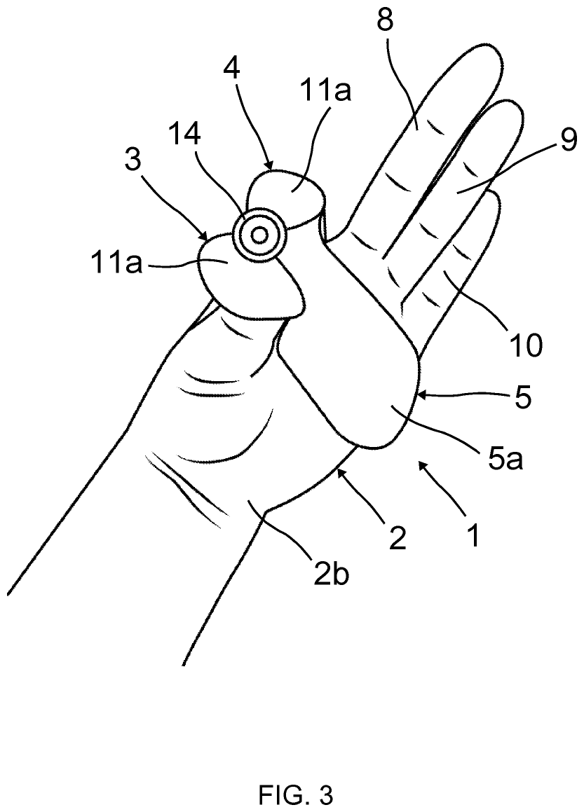

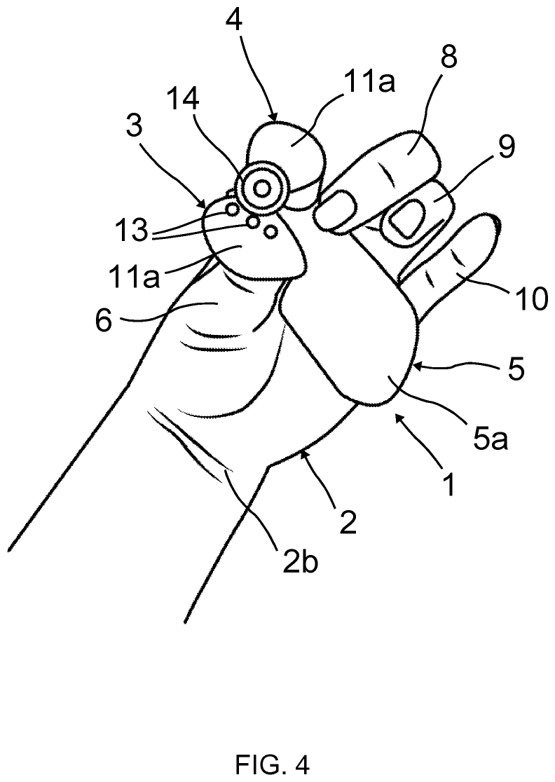

The design of the protective device 1 is based on the typical hand movements performed when closing or re-closing pointed and sharp medical utensils, such as cannulas, lancets, scalpels and the like. The securing or closing elements, which may be sheaths or caps 14, for example, are generally held between the thumb 6 and index finger 7 (cf. FIGS. 3 and 4) while the previously used instrument is carefully inserted. The applicant has found that in this process the fingertips of the two fingers 6, 7 used for holding are particularly at risk of being injured, as is the palm of the user's hand. The protective device 1 reliably protects precisely these particularly vulnerable areas of the hand 2 during the reclosing process. At the same time, the protective device 1 can be applied very conveniently and particularly quickly by a user. He only has to push the tips of his thumb 6 and index finger 7 into the finger protection caps 11 of the finger protection elements 3, 4. The palm protection element 5 connecting the two finger protection elements 3, 4 then lies protectively in front of his palm. Since the protective device 1 is made of an elastic material, the finger guards 3, 4 can be conveniently brought together, for example, to grip a cap 14. The finger protectors 12 and the palm protector 5 are then bent accordingly, which can be seen in particular in FIG. 5.

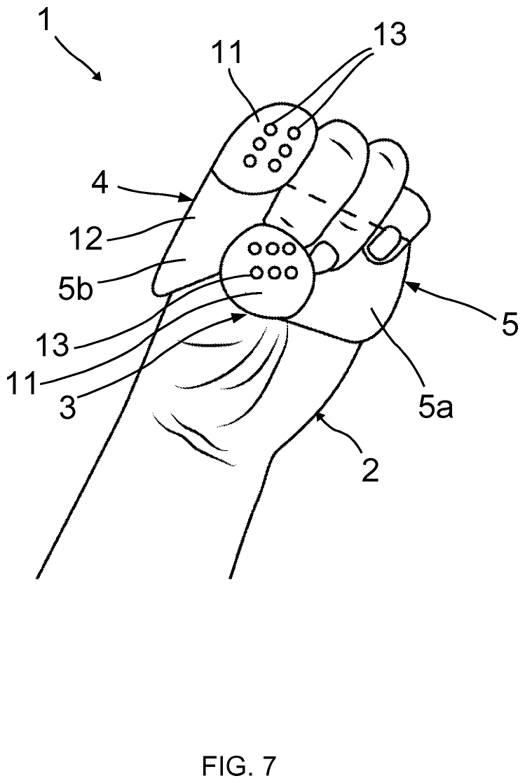

The user can optionally slide those fingers that are not covered by a finger protection element 3, 4, i.e. the middle finger 8, ring finger 9 and little finger 10, or their front sections, behind the palm protection element 5 in order to protect them as well. This is schematically indicated in FIGS. 6 and 7.

Since the protective device 1 can be put on particularly quickly, especially in comparison with previously known full as well as partial gloves which have to be pulled over the hand and up to the wrist in a relatively laborious manner, the effort associated with disposal is minimized. If the effort of proper as well as safe disposal is low, users are more likely to opt for it and the risk of health hazards is reduced. The protective device 1 can thereby also be used in combination with thin disposable rubber gloves, which are often worn in the medical or nursing field. These do not have to be removed before the protective device 1 is put on by inserting the thumb 6 and index finger 7, or tips thereof.

I claim:

1. Protective device (1) for a hand (2), which comprises precisely two finger protection elements (3, 4) and a palm protection element (2) connecting the two finger protection elements (3, 4), wherein one finger protection element (3) is intended for the thumb (6) and the other finger protection element (4) is adapted for a further finger, in particular the index finger (7), of a user, and the palm protection element (2) is configured to be planar, so that it only comes into contact with the palm of a user's hand, whereas the back of the user's hand remains free or essentially free.

2. Protective device (1) according to claim 1, wherein the finger protection elements (3, 4) both lie closer to one end region (5b) of the palm protection element (5) than to another end region (5a) of the palm protection element (5) opposite the one end region (5b), in particular, wherein the finger protection elements (3, 4) both lie directly at the one end region (5b) of the palm protection element (5), and/or wherein the other end region (5a) is a free end region of the palm protection element (5).

3. Protective device (1) according to claim 2, wherein the palm protective element (5) has a preferably free end region with a U-shaped outer contour, tip region (5a), and an end region opposite the tip region (5a), root region (5b), the finger protective elements (3, 4) both lying closer to the root region (5b) than to the tip region (5a), in particular, the finger protection elements (3, 4) both lying directly on the root region (5b), preferably, the root region (5b) terminating with an edge (5c), in particular a straight edge, opposite the tip region (5a), and the finger protection elements (3, 4) extending up to this edge (5c).

4. Protective device (1) according to claim 2, wherein the finger protection elements (3, 4) are arranged laterally of the palm protection element (5) with respect to the two opposite end regions (5a, 5b), preferably projecting laterally from the palm protection element (5).

5. Protective device (1) according to claim 4, wherein one of the finger protection elements (3, 4) is arranged on each of two opposite sides of the palm protection element (5), and/or wherein the finger protection elements (3, 4) are open at one end and closed at their end opposite the open end, the open end facing the palm protection element (5) and the closed end facing away from the palm protection element (5).

6. Protective device (1) according to claim 1, wherein the finger protection elements (3, 4) each comprise a finger protection cap (11) with a closed upper side (11a) and an open lower side (11b), preferably wherein the finger protection caps (11) have at least substantially the shape of a hollow truncated cone or at least substantially the shape of a hollow cylinder closed on one side, and/or wherein the finger protection caps (11) are characterized by a greater material thickness in the region of their closed upper sides (11a) than in the remaining region.

7. Protective device (1) according to claim 1, wherein the finger protection elements (3, 4) each have a finger protection web (12), in particular, wherein the finger protection web (12) of the finger protection element (3) intended for the thumb (6) has a shorter length than the finger protection web (12) of the finger protection element (4) intended for the further finger (7) and/or wherein the material thickness of the finger protection webs (12), at least in the region of the front ends associated with the fingertips, is at least 1.0 mm, in particular at least 2 mm, and/or is at most 5.0 mm, in particular at most 4.0 mm, and is preferably 3.0±0.5 mm.

8. Protective device (1) according to claim 6, wherein the finger protection web (12) is connected to the open underside (11b) of the finger protection cap (11).

9. Protective device (1) according to claim 6, wherein nubs (13) are provided on the inside and/or the outside of the finger protection caps (11).

10. Protective device (1) according to claim 7, wherein,
the material thickness of the finger protection caps (11) is at least 1.0 mm, in particular at least 2 mm, at least in sections, and/or wherein the material thickness of the finger protection caps (11) is at most 5.0 mm, in particular at most 4.0 mm, and is preferably 3.0±0.5 mm, and/or
the interior of the finger protection caps (11) has a diameter of 1.5 to 3.0 cm, in particular at the widest point, and/or wherein the interior of the finger protection caps (11) has a length in the range from 1.5 to 6.0 cm, and/or
wherein the finger protection caps (11) have a length of at least 2 cm, in particular at least 2.5 cm, and/or of at most 4.0 cm, in particular at most 3.0 cm.

11. Protective device (1) according to claim 1, wherein the finger protection elements (3, 4) are connected to each other exclusively via the palm protection element (5).

12. Protective device (1) according to claim 1, wherein
the palm protection element (5) is designed as a flexible mat, and/or
the palm protection element (5) is designed and arranged in such a way that, in the state in which the protective device (1) is worn as intended, when the two finger protection elements (3, 4) are each placed on a finger, in particular the thumb (6) and index finger (7), of a user, it extends protectively in front of the palm of the user's hand.

13. Protective device (1) according to claim 1, wherein
the palm protection element (5) has a thickness of at least 1.0 mm, in particular at least 2 mm, and/or a maximum of 5.0 mm, in particular a maximum of 4 mm, the thickness preferably being 3.0±0.5 mm, and/or
the palm protection element (5) has a length of at least 6.0 cm, in particular at least 7.0 cm and/or of at most 11.0 cm, in particular at most 9.0±0.5 cm.

14. Protective device (1) according to claim 1, wherein the finger protection elements (3, 4) and/or the palm protection element (5) comprise or consist of at least one elastically deformable material, in particular at least one elastomer and/or silicone, wherein in particular the material is reinforced and preferably reinforcing elements in the form of strips of metal sheet, plastic or reinforcing fibers are embedded in the material.

15. Protective device (1) according to claim 1, wherein the protective device (1) is designed in one piece, and/or wherein the protective device (1) is designed in such a way that it leaves three fingers, preferably the ring finger (8), middle finger (9) and little finger (10), and/or the back of the hand (2a) and/or the wrist (2b) free.

16. Protective device (1) according to claim 3, wherein the finger protection elements (3, 4) are arranged laterally of the palm protection element (5) with respect to the two opposite end regions (5a, 5b), preferably projecting laterally from the palm protection element (5).

17. Protective device (1) according to claim 2, wherein the finger protection elements (3, 4) each comprise a finger protection cap (11) with a closed upper side (11a) and an open lower side (11b), preferably wherein the finger protection caps (11) have at least substantially the shape of a hollow truncated cone or at least substantially the shape of a hollow cylinder closed on one side, and/or wherein the finger protection caps (11) are characterized by a greater material thickness in the region of their closed upper sides (11a) than in the remaining region.

18. Protective device (1) according to claim 3, wherein the finger protection elements (3, 4) each comprise a finger protection cap (11) with a closed upper side (11a) and an open lower side (11b), preferably wherein the finger protection caps (11) have at least substantially the shape of a hollow truncated cone or at least substantially the shape of a hollow cylinder closed on one side, and/or wherein the finger protection caps (11) are characterized by a greater material thickness in the region of their closed upper sides (11a) than in the remaining region.

19. Protective device (1) according to claim 4, wherein the finger protection elements (3, 4) each comprise a finger protection cap (11) with a closed upper side (11a) and an open lower side (11b), preferably wherein the finger protection caps (11) have at least substantially the shape of a hollow truncated cone or at least substantially the shape of a hollow cylinder closed on one side, and/or wherein the finger protection caps (11) are characterized by a greater material thickness in the region of their closed upper sides (11a) than in the remaining region.

20. Protective device (1) according to claim 5, wherein the finger protection elements (3, 4) each comprise a finger protection cap (11) with a closed upper side (11a) and an open lower side (11b), preferably wherein the finger protection caps (11) have at least substantially the shape of a hollow truncated cone or at least substantially the shape of a hollow cylinder closed on one side, and/or wherein the finger protection caps (11) are characterized by a greater material thickness in the region of their closed upper sides (11a) than in the remaining region.
